(19) United States
(12) Patent Application Publication
(10) Pub. No.: US 2012/0100206 A1
(43) Pub. Date: Apr. 26, 2012

(54) TARGETED LIPOSOMES COMPRISING N-CONTAINING BISPHOSPHONATES AND USES THEREOF

(75) Inventors: Hilary Shmeeda, Givat Zeev (IL); Alberto A. Gabizon, Jerusalem (IL)

(73) Assignee: Yissum Research Development Company of the Hebrew University of Jerusalem, Ltd., Israel (IL)

(21) Appl. No.: 13/377,629

(22) PCT Filed: Jun. 10, 2010
(86) PCT No.: PCT/IL2010/000464

§ 371 Date: Dec. 12, 2011

Related U.S. Application Data
(60) Provisional application No. 61/186,042, filed on Jun. 11, 2009.

(51) Int. Cl. A61K 9/127; A61K 31/675; A61P 19/10; A61K 31/663

(52) U.S. Cl. 424/450; 514/114; 514/89

(57) ABSTRACT

The present is based on the finding that folate targeted liposomal alendronate (FT-AL-L) was significantly more potent against two tested cancer cell lines than the free alendronate (AL) or the non-targeted liposomal alendronate (AL-L), as observed by the increased cytotoxicity of the folate targeted liposomal alendronate. Thus, the present disclosure provides targeted liposomes comprising a membrane and an intraliposomal core, the membrane comprising at least one liposome forming lipid and a targeting moiety, such as folate, exposed at the membrane's outer surface; and the intraliposomal core comprising encapsulated therein least one N-containing bisphosphonate. Also provided by the present disclosure are methods of use of the targeted liposomes such as for the treatment of a disease or disorder.

### FIELD OF THE INVENTION

This invention relates to targeted liposomes comprising N-containing bisphosphonates and uses thereof in therapy.

### BACKGROUND OF THE INVENTION

Bisphosphonates are drugs consisting of two known classes, the simple bisphosphonates (clodronate, etidronate) and the N-containing-bisphosphonates (also known as amino-bisphosphonates) such as tiludronate, alendronate, pamidronate, ibandronate, neridronate, risedronate and zoledronate. The simple bisphosphonates are metabolized to non-hydrolyzable analogs of adenosine triphosphate and diadenosine tetraphosphates, whereas the N-containing-bisphosphonates are potent inhibitors of farnesyl diphosphate synthase, one of the major enzymes of the mevalonate pathway.

Bisphosphonates are used primarily to increase bone mass and reduce the risk of fracture in patients with osteoporosis, to slow bone turnover in patients with Paget's disease of the bone, and to treat bone metastases and normalize elevated levels of blood calcium in patients with cancer [Green J. R. Biophosphonates: preclinical review, Oncologist 8 (suppl 4) (2004) 3-13]. Zoledronic acid and other N-containing bisphosphonates have also been found to interfere with critical processes in cell signaling and growth at nanomolar concentrations and are currently under evaluation for use in combination therapies for various anti-tumor applications [Coleman R and Gnant M, new results from the use of bisphosphonates in cancer patients, Curr. Opin. Support. Palliat. Care 3(3) (2009) 213-218]. In addition to anti-tumor effect, anti-angiogenic effects have also been demonstrated [Santini D, Schiavon G, Angeletti S, Vincenzi B, Gasparro S, Grilli C, La Cesa A, Virzí V, Leoni V, Budillon A, Addeo S R, Caraglia M, Dicuonzo G, Tonini G. Last generation of amino-bisphosphonates (N-BPs) and cancer angio-genesis: a new role for these drugs? Recent Pat Anticancer Drug Discov. 2006 November; 1(3):383-96.]. However, these bisphosphonates have very low cellular permeability and this substantially limits their anti tumor efficacy.

Bisphosphonate-liposomes formulations have been described, for example, in US application publication No. 2007/0218116 which describes a method for treating or preventing tumor growth and metastasis by administrating liposomal bisphosphonates.

In addition, US patent application publication No. 2004/0161457 describes a method for administrating a therapeutic compound encapsulated in liposome to multi-drug resistant cancer cells. This method also included a covalently attached folate (folic acid) ligand to the liposome carrier.

Further, the synthesis and in vitro as well as in vivo studies of folate targeted PEG as potential carrier for anti-cancer drugs was also described [Aronov O, Horowitz A T, Gabizon A, and Gibson D: “Folate targeted PEG as potential carrier for carboplatin analogs: Synthesis and in vitro studies.” Bioconjugate Chemistry, 14:563-574, 2003; Gabizon A, Horowitz A T, Goren D, Tzemach D, Shmeeda H, and Zalipsky S: “In vivo fate of folate-targeted polyethylene-glycol liposomes in tumor-bearing mice.” Clinical Cancer Research, 9:6551-6559, 2003; Shmeeda H, Mak L, Tzemach D, Astrahan P, Tarshish M, and Gabizon A: “Intracellular uptake and intracavitary targeting of folate-conjugated liposomes in a mouse lymphoma model with upregulated folate receptors.” Molecular Cancer Therapeutics 5:818-824, 2006]

### SUMMARY OF THE INVENTION

The present disclosure provides, in accordance with a first of its aspects, targeted liposomes comprising a membrane and an intraliposomal core, the membrane comprising at least one liposome forming lipid and a targeting moiety exposed at the membrane's outer surface; and the intraliposomal core comprising encapsulated therein least one N-containing bisphosphonate.

In accordance with a second aspect, the present disclosure provides the use of targeted liposomes for the treatment of a disease or disorder.

In accordance with yet a further aspect, the present disclosure also provides a method of treatment comprising administering to a subject in need of treatment an amount of the targeted liposomes disclosed herein.

Further, the present disclosure provides a pharmaceutical composition comprising as active ingredient targeted liposomes as disclosed herein, in combination with a physiologically acceptable carrier.

In one particular embodiment, the targeted liposomes comprise folate (folic acid) as the targeting moiety.

In yet another embodiment, the targeted liposomes comprise folate as the targeting moiety in combination with alendronate as the N-containing bisphosphonate.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 is a graph showing the cytotoxicity effect of free alendronate (AL), liposomal alendronate (AL-L) and folate-targeted liposomal alendronate (FT-AL-L), as measured in IGROV-1HiFR (human ovarian carcinoma with high expression of folate-receptor) cells; the graph shows that the folate-targeted liposomal alendronate had increased cytotoxicity against the cancer cells indicating that alendronate is delivered into cells by folate-targeted liposomes more effectively than as free drug.

FIG. 2 is a graph showing the cytotoxicity effect of free alendronate (AL), liposomal alendronate (AL-L) and folate targeted liposomal alendronate (FT-AL-L), as measured in KB-HiFR (human head-and-neck carcinoma with high expression of folate-receptor) cells; the graph shows that the folate targeted liposomal alendronate had increased cytotoxicity against the cancer cells indicating that alendronate is delivered into cells by folate-targeted liposomes more effectively than as free drug.

FIG. 3 is a graph showing dose escalation study for determination of the maximum tolerated dose (MTD) of liposomal alendronate as determined by cumulative doses in four Balb/C mice; the graph shows that no toxicity was detected at any of the tested administered doses of this specific N-containing bisphosphonates.

### DETAILED DESCRIPTION OF EMBODIMENTS

The present disclosure is based on the finding that while bisphosphonate have low permeability into cells, it is possible to significantly increase the permeability and cytotoxicity of liposomal N-containing bisphosphonates by providing at their outer surface a targeting moiety, such as folate, for targeting delivery of the liposomal N-containing bisphosphonate to cells expressing and presenting at their surface the target receptor, such as folate receptor. This finding led to the development of “targeted liposomal N-containing bisphosphonates” disclosed herein.

Thus, in accordance with the first aspect of the present disclosure, there is provided a population of liposomes carrying at their outer surface a targeting moiety.

Specifically, the present disclosure provides targeted liposomes, each liposome comprising a membrane and an intraliposomal core, the membrane comprising at least one liposome forming lipid and a targeting moiety exposed at the membrane's outer surface; and the intraliposomal core comprising encapsulated therein least one N-containing bisphosphonate.

In one embodiment, the mole:mole ratio between the N-containing bisphosphonate and the lipid is between 0.1 and 1.5, at times 0.8 and 1.3.

The liposomes comprise at least one liposome forming lipid, typically at least one phospholipid, forming the liposomes' bilayer membrane which encloses the intraliposomal aqueous phase/core. The term “liposome forming lipids” as used herein denotes those lipids having a glycerol backbone wherein at least one, preferably two, of the hydroxyl groups at the head group is substituted by one or more of an acyl, an alkyl or alkenyl chain, a phosphate group, preferably an acyl chain (to form an acyl or diacyl derivative), a combination of any of the above, and/or derivatives of same, and may contain a chemically reactive group (such as an amine, acid, ester, aldehyde or alcohol) at the headgroup, thereby providing a polar head group. Typically, a substituting chain, e.g. the acyl, alkyl or alkenyl chain, is between about 14 to about 24 carbon atoms in length, and has varying degrees of saturation, thus resulting in fully, partially or non-hydrogenated (liposome-forming) lipids.

Further, the lipids may be of a natural source, semi-synthetic or a fully synthetic lipid, and may be neutral, negatively or positively charged. There are a variety of synthetic vesicle-forming phospholipids and naturally-occurring vesicle-forming phospholipids, including the phospholipids, such as phosphatidylcholine (PC), phosphatidylinositol (PI), phosphatidylglycerol (PG), dimyristoyl phosphatidylglycerol (DMPG); egg yolk phosphatidylcholine (EPC), 1-palmitoyl-2-oleoylphosphatidyl choline (POPC), distearoylphosphatidylcholine (DSPC), dimyristoyl phosphatidylcholine (DMPC); phosphatidic acid (PA), phosphatidylserine (PS); 1-palmitoyl-2-oleoylphosphatidyl choline (POPC), and the sphingophospholipids such as sphingomyelins (SM) having 12- to 24-carbon atom acyl or alkyl chains.

Lipids having a relatively high Tₘ may be referred to as “rigid” lipids, typically those having saturated, long acyl chains, while lipids with a relatively low Tₘ may be referred to as “fluid” lipids. Fluidity or rigidity of the liposome may be determined by selecting lipids with pre-determined fluidity/rigidity for use as the liposome-forming lipids. In accordance with one embodiment, the Tₘ of the lipids forming the liposomes is preferably equal to or above 30° C. at times even equal to or above 40° C.

A non limiting example of lipids forming the liposomes and having a Tₘ above 30° C. comprises phosphatidylcholine (PC) and derivatives thereof having two acyl (or alkyl) chains with 16 or more carbon atoms. Some preferred examples of PC derivatives which form the basis for the low permeable liposomes in the context of the invention include, without being limited thereto, hydrogenated soy PC(HSPC) having a Tₘ of 52° C., Dipalmitoylphosphatidylcholine (DPPC), having a Tₘ of 41.3° C., N-palmitoyl sphingomyelin having a Tₘ of 41.2° C., distearylphosphatidylcholine (DSPC) having a Tₘ of 55° C., N-stearoyl sphingomyelin having a Tₘ of 48° C., distearyolphosphatidylglycerol (DSPG) having a Tₘ of 55° C., partially hydrogenated phosphatidyl-choline (PHPC) having a Tₘ of 35° C. and distearyphosphatidylserine (DSPS) having a Tₘ of 68° C.

All the above Tₘ temperature data are from http://www.avantilipids.com Phase Transition Temperatures or from http://www.lipidat.tcd.ie., as known to those versed in the art. Those versed in the art will know how to select a lipid with a Tₘ either equal or above 25° C., 30° C. or even equal or above 40° C. [see also Barenholz, Y., Liposome application: problems and prospects. Curr. Opin. Colloid Interface Sci. 6, 66-77 (2001); Barenholz, Y. and Cevc, G., Structure and properties of membranes. In Physical Chemistry of Biological Surfaces (Baszkin, A. and Norde, W., eds.), Marcel Dekker, NY (2000) pp. 171-2411

The liposomes may further comprise membrane active sterols (e.g. cholesterol) and/or phosphatidylethanolamines in order to decrease a membrane's free volume and thereby permeability and leakage of material loaded therein. In one embodiment, the membrane comprises cholesterol. The addition of sterol is known to affect permeability of the liposomes.

Further, the liposomes may also include a lipid derivatized with a hydrophilic polymer to form new entities known by the term lipopolymers. Lipopolymers preferably comprise lipids (liposome forming lipids as well as lipids that do not from into lipids, such as phosphatidylethanolamines) modified at their head group with a polymer having a molecular weight equal to or above 750 Da. The head group may be polar or apolar; however, it is preferably a polar head group to which a large (>750 Da), highly hydrated (at least 60 molecules of water per head group), flexible polymer is attached. The attachment of the hydrophilic polymer head group to the lipid region may be a covalent or non-covalent attachment; however, it is preferably via the formation of a covalent bond (optionally via a linker). The outermost surface coating of hydrophilic polymer chains is effective to provide a liposome with a long blood circulation lifetime in vivo.

Examples have been described in Tirosh et al. [Tirosh et al., Biopys. J., 74(3):1371-1379, (1998)] and in U.S. Pat. Nos. 5,013,556; 5,395,619; 5,817,856; 6,043,094; and 6,165,501; incorporated herein by reference; and in WO 98/07409. The lipopolymers may be non-ionic lipopolymers (also referred to at times as neutral lipopolymers or uncharged lipopolymers) or lipopolymers having a net negative or a net positive charge.

There are numerous polymers which may be attached to lipids. Polymers typically used as lipid modifiers include, without being limited thereto: polyethylene glycol (PEG), polysialic acid, polylactic acid (also termed polylactide), polyglycolic acid (also termed polyglycolide), polylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose. The polymers may be employed as homopolymers or as block or random copolymers.

The lipopolymer may be introduced into the liposome in two different ways either by: (a) adding the lipopolymer to a lipid mixture, thereby forming the liposome, where the lipopolymer will be incorporated and exposed at the inner and outer leaflets of the liposome bilayer [Uster P. S. et al. FEBBS Letters 386:243 (1996)]; or (b) first preparing the liposome and then incorporating the lipopolymers into the external leaflet of the pre-formed liposome either by incubation at a temperature above the Tₘ of the lipopolymer and liposome-forming lipids, or by short-term exposure to microwave irradiation.

While the lipids derivatized into lipopolymers may be neutral, negatively charged, or positively charged, i.e. there is no restriction regarding a specific (or no) charge, the most commonly used and commercially available lipids derivatized into lipopolymers are those based on phosphatidyl ethanolamine (PE), usually, distearylphosphatidylethanolamine (DSPE).

A specific family of lipopolymers which may be employed by the invention include monomethylated PEG attached to DSPE (with different lengths of PEG chains, the methylated PEG referred to herein by the abbreviation PEG) in which the PEG polymer is linked to the lipid via a carbamate linkage resulting in a negatively charged lipopolymer. Other lipopolymer are the neutral methyl polyethyleneglycol distearoylglycerol (mPEG-DSG) and the neutral methyl polyethyleneglycol oxycarbonyl-3-amino-1,2-propanediol distearoylester (mPEG-DS) [Garbuzenko O. et al., Langmuir. 21:2560-2568 (2005)]. The PEG preferably has a molecular weight of the PEG head group is from about 750 Da to about 20,000 Da. More preferably, the molecular weight is from about 750 Da to about 12,000 Da, and it is most preferably between about 1,000 Da to about 5,000 Da. One specific PEG-DSPE employed herein is a PEG moiety with a molecular weight of 2000 Da, designated herein ²⁰⁰⁰PEG-DSPE or ^{2k}PEG-DSPE.

In liposomes including such derivatized lipids it typically includes between 1-20 mole percent of such a derivatized lipid is included in the liposome formulation.

The liposome may include other constituents. For example, charge-inducing lipids, such as phosphatidylglycerol, such as dipalmitoylphosphatidylglycerol (DPPG, Tₘ of about 41° C.) may also be incorporated into the liposome bilayer to decrease vesicle-vesicle fusion, and to increase interaction with cells. Buffers at a pH suitable to make the liposome surface's pH close to neutral can decrease hydrolysis. Addition of an antioxidant, such as vitamin E, or chelating agents, such as Desferal or DTPA, may be used.

The liposomes according to the present disclosure are typically those having a diameter of between about 70 nm to 130 nm, or even between about 80 nm and 110 nm. The liposomes may be unilamellar, bilamellar or even, at times, multilamellar. In one embodiment, the liposomes are thus small unilamellar vesicles (SUV), although the population of liposomes may include also some liposomes with more than one lamella.

With respect to the N-containing bisphosphonate some are those with a common PX₃-CR₁R₂-PX₃ backbone, where X is either H or —OH. The N-containing bisphosphonate are those carrying N-containing substituents at R₁ and/or R₂, such as those presented in the following Table 1:

TABLE 1
N-containing bisphosphonates

| Common name | R₁ | R₂ |
|---|---|---|
| Alendronate | —OH | —(CH₂)₃—NH₃ |
| Pamidronate | —OH | —(CH₂)₂—NH₃ |
| neridronate | —OH | —(CH₂)₅—NH₃ |
| Olpadronate | —OH | —(CH₂)₂N(CH₂)₂ |
| Ibandronate | —OH | —(CH₂)₂N(CH₃)(CH₂)₄CH₃ |
| Risedronate | —OH | 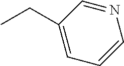 |

The above N-containing bisphosphonates are also known by the following nomenclature:

Alendronate—alendronic acid, 4-amino-1-hydroxybutylidene-1,1-bisphosphonic acid, alendronate sodium or monosodium trihydrate; described in U.S. Pat. No. 4,922,007 and U.S. Pat. No. 5,019,651, both of which are incorporated by reference herein in their entirety);

Ibandronate—1-hydroxy-3-(N-methyl-N-pentylamino) propylidene-1,1-bisphosphonic acid, also known as BM-210955, described in U.S. Pat. No. 4,927,814, which is incorporated by reference herein in its entirety;

Neridronate—6-amino-1-hydroxyhexylidene-1,1-bisphosphonic acid;

Olpadronate—3-(dimethylamino)-1-hydroxypropylidene-1,1-bisphosphonic acid;

Pamidronate—3-amino-1-hydroxypropylidene-1,1-bisphosphonic acid;

Risedronate—1-hydroxy-2-(3-pyridinyl)-ethylidene-1,1-bisphosphonic acid;

Other N-containing bisphosphonates are [2-(2-pyridinyl)ethylidene]-1,1-bisphosphonic acid (piridronate, described in U.S. Pat. No. 4,761,406, which is incorporated by reference in its entirety); 4-chlorophenyl)thiomethane-1,1-disphosphonic acid (tiludronate, described in U.S. Pat. No. 4,876,248, incorporated herein by reference, in its entirety).

The N-containing bisphosphonate also include pharmaceutically acceptable salts and derivatives thereof. As used herein, the terms “pharmaceutically acceptable” refer to salts of the N-containing bisphosphonate that are “generally regarded as safe” (GRAS), e.g., that are physiologically tolerable and do not typically produce an allergic or similar untoward reaction, such as gastric upset, dizziness and the like, when administered to an animal. Preferably, as used herein, the term “pharmaceutically acceptable” means approved by a regulatory agency of the Federal or a state government or listed in the U.S. Pharmacopeia or other generally recognized pharmacopeia for use in animals. Non-limiting examples of salts include those selected from the group consisting alkali metal, alkaline metal, ammonium, and mono-, di, tri-, or tetra-Q-Cso-alkyl-substituted ammonium. Some particular salts are those selected from the group consisting of sodium, potassium, calcium, magnesium, and ammonium salts. Non-limiting examples of derivatives include those selected from the group consisting of esters, hydrates, and amides.

The liposome comprise the targeting moiety exposed at least partially at the liposome's outer surface. The targeting moiety may be any ligand that can associate (covalently or non-covalently) to the outer surface of the liposome and have affinity to a target tissue or target organ. Some non-limiting targeting moieties include folate, Luteinizing-hormone-releasing hormone (LH-RH); the growth inhibiting hormone, somatostatin, the blood plasma protein, transferrin; target specific antibody such as anti Her2, anti EGFr, anti nucleosome. The targeting moiety is typically between 0.1-0.5% out of the total lipid content in the liposome.

A particular embodiment of the present disclosure concerns folate targeted liposomes which are targeted to cells expressing folate receptor such as types of cancer cells. In one embodiment, the cancer is solid cancer, such as, without being limited thereto, brain, breast, prostate, colorectum, kidney; sarcoma; melanoma.

In one embodiment, the targeted liposomes comprise a liposome forming lipid (one or more), cholesterol and the folate-conjugate. In one particular embodiment, the molar ratio between the aforementioned components is, from 55:40:5, without being limited thereto.

The targeted liposomes may be prepared by any method known to those versed in the art of liposomes. On one embodiment, the targeted liposomes are prepared by using a conjugate between the targeting moiety and a membrane forming component, such as a lipopolymer. The conjugate may be mixed with liposome forming lipids to form the targeted liposome or it may be mixed (incubated) with pre-formed liposomes under conditions which permit the incorporation of the lipopolymer portion of the conjugate into the liposome's membrane. In one embodiment, the conjugate is a conjugate of folate and PEG-lipid, such as folate-^{2k}PEG-DSPE or folate-^{3.35k}PEG-DSPE.

In one embodiment, the liposomes encapsulating the N-containing bisphosphonate are formed by rehydrating the liposome-forming lipids with a solution of the N-containing bisphosphonate at a temperature above the Tm of the liposome forming lipids. This process typically achieves passive encapsulation of the N-containing bisphosphonate in the intra-liposomal water phase and downsizing the preformed liposomes to the desired dimensions. Downsizing may be achieved, for example, by extrusion through polycarbonate membranes using an extruder with a pre-selected pore size.

The final liposome sizes are typically 70-130 nm as measured, and at times 80 nm-110 nm, depending, inter alia, on the pore size used during downsizing.

The non-encapsulated N-containing bisphosphonate may then be removed by dialysis and/or use of an adequate anion-exchange resin.

In accordance with the present disclosure, the liposomes may be used for the treatment of a disease or disorder. In one embodiment, the disease or disorder is such for which the N-containing bisphosphonate is therapeutically effective.

In one embodiment, the disease or disorder is a proliferative disease.

In yet a further embodiment, the disease or disorder is cancer.

The cancer may be a type for which N-containing bisphosphonate are known to be effective, such as secondary bone cancer (bone metastasis).

Further provided by the present disclosure are pharmaceutical compositions comprising as active ingredient the liposomes defined herein in combination with a physiologically acceptable carrier.

Yet further provided by the present disclosure is a method for treating a disease or disorder, e.g. a proliferative disease or disorder, comprising administering to a subject in need an amount of the targeted liposomes as defined herein.

The liposomes may be formulated in any form suitable for administration of anti-proliferative drugs.

The term “administering” (“administration”) is used to denote the contacting or dispensing, delivering or applying of the targeted liposomes to a subject by any suitable route of delivery thereof to the desired location in the subject, including parenteral (including subcutaneous, intramuscular and intravenous, intra-arterial, intraperitoneal, etc.) and intranasal administration, as well as intrathecal and infusion techniques.

According to one embodiment, the liposomes are formulated in a form suitable for injection. The requirements for effective pharmaceutical vehicles for injectable formulations are well known to those of ordinary skill in the art [See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986)].

As used herein the term “treatment” (or “treating”) denotes curing of an undesired disease or disorder or prevention of a disease or disorder from developing. For the purpose of curing, the term “treatment” includes direct effect on the causative of the diseases, such as reducing tumor load, preventing cancer related cells from proliferating, etc, as well as indirect effect, e.g. for ameliorating undesired symptoms associated with the gene, slowing down progression of the condition, delaying the onset of a progressive stage of the condition, slowing down deterioration of such symptoms, enhancing onset of a remission period of the disease or disorder, if existing, delaying onset of a progressive stage, improving survival rate or more rapid recovery from the disease or disorder, lessening the severity of or curing the disease or disorder, etc. Treatment also includes prevention of a disease or disorder. The term “prevention” includes, without being limited thereto, administering an amount of the composition to prevent the disease or disorder from developing or to prevent irreversible damage caused by the disease or disorder, to prevent the manifestation of symptoms associated with the disease or disorder before they occur, to inhibit the progression of the disease or disorder etc.

The pharmaceutical composition may be provided as a single dose, or in several doses to be administered more than once a day, for an extended period of time (e.g. to produce cumulative effective amount) in a single daily dose for several days, in several doses a day, etc.

The treatment regimen and the specific formulation of the targeted liposomes to be administered will depend on the type of disease or disorder to be treated and may be determined by various considerations, known to those skilled in the art of medicine, e.g. physicians. The term “amount effective for” or similar is used herein to denote the amount of the N-containing bisphosphonate, which, when loaded into the liposome, is sufficient in a given therapeutic regimen to achieve a desired therapeutic effect with respect to the treated disease or disorder. The amount is determined by such considerations as may be known in the art and depends on the type and severity of the condition to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors, including the mode of administration, type of liposome carrying the N-containing bisphosphonate, the reactivity of each of the N-containing bisphosphonate, the liposome's distribution profile within the body, a variety of pharmacological parameters such as half-life in the body after being released from the liposome, undesired side effects, if any, factors such as age and gender of the treated subject, etc.

It is noted that the forms “a”, “an” and “the” as used in the specification include singular as well as plural references unless the context clearly dictates otherwise. For example, the term “a lipid” or “a targeted liposome” includes one or more, of the same or different lipids as well as one or more such targeted liposomes.

Similarly, reference to the plural includes the singular, unless the context clearly dictates otherwise.

Further, as used herein, the term “comprising” is intended to mean that the liposome includes the recited constituents, but does not exclude others which may be optional in the formation or composition of the liposome, such as antioxidants, cryoprotectants, etc. The term “consisting essentially” of is used to define a substance, e.g. liposome, that includes the recited constituents but excludes other constituents that may have an essential significant effect on a parameter of the liposomes, the stability, release or lack of release of the agent from the liposome as well as on other parameters characterizing the liposomes); “consisting of” shall thus mean excluding more than trace amounts of such other constituents. Embodiments defined by each of these transition terms are within the scope of this invention.

Further, all numerical values, e.g. when referring the amounts or ranges of the elements constituting the composition or liposome components, are approximations which are varied (+) or (−) by up to 20 percent, at times by up to 10 percent from the stated values. It is to be understood, even if not always explicitly stated, that all numerical designations are preceded by the term “about”. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. Although the invention has been described in conjunction with specific embodiments thereof, it is evident that many alternatives, modifications and variations will be apparent to those skilled in the art, and it is explicitly intended that the invention include such alternatives, modifications and variations.

All publications, patents and patent applications mentioned in this specification are herein incorporated in their entirety by reference into the specification.

### DESCRIPTION OF SOME NON-LIMITING EXAMPLES

### Materials

Hydrogenated soybean phosphatidyl-choline (HSPC) (Lipoid, Germany)
Partially hydrogenated phosphatidyl-choline (PHPC), (Lipoid, Germany)
mPEG(2000)-DSPE (Bio-lab, Jerusalem, Israel);
Cholesterol (Sigma, St. Louis, Mo.)

### Alendronate (Teva, Israel)

BALB/c mice (Harlan, Israel)
KB-HiFR human head and neck carcinoma expressing high folate receptor (FR) (Beit HaEmek, Israel).
IGROV-1-HiFR human ovarian carcinoma expressing high FR (Beit HaEmek, Israel).

### Methods

### Liposomal Formulation:

Liposome encapsulation was performed by standard methods of lipid lyophilization, hydration and polycarbonate membrane extrusion down to 0.05 μm pore size

Briefly, the phospholipids used were hydrogenated soybean phosphatidyl-choline (HSPC) and mPEG (2000)-DSPE, with or without cholesterol.

The lipid components were used at the following mole ratios; 55% HSPC; 40% cholesterol; 5% ^{2k}pEG-DSPE were weighed, dissolved in tertiary-butanol, frozen in liquid nitrogen and lyophilized overnight.

### Alendronate Encapsulation:

Alendronate was loaded into the liposomes by rehydration the lyophilized lipids in a solution comprising 15 mM histidine pH 7.0 buffer in 5% dextrose: 0.9% saline (9:1 volume ratio) and 100 mM alendronate. Re-suspended liposomes were processed by serial size extrusion in a high-pressure extruder device (Lipex Biomembranes, Vancouver, BC) with temperature control through filters with pore sizes from 1000 nm to 50 nm. For PHPC-based formulations, the temperature was set at 35° C.-40° C. and for HSPC-based formulations, the temperature was set at 60-65° C.

Non-encapsulated bisphosphonate was removed by dialysis followed by passage through a small column with Dowex anion exchange resin (1×2-400 beads (Sigma). The liposomes were sterilized by filtration through 0.22 μM filters and stored in Vacutainer™ tubes at 4° C.

Phospholipid and alendronate content were determined by Bartlett phosphorous assay of Folch extracted samples (8:4:3 chloroform:methanol:DDW) [Shmeeda, H., Even-Chen, S., Honen, R., Cohen, R., Weintraub, C., and Barenholz, Y. Enzymatic assays for quality control and pharmacokinetics of liposome formulations: comparison with nonenzymatic conventional methodologies. Methods Enzymol, 367: 272-292, 2003]

Liposome size and Zeta potential were determined using a NanoZ (Malvern Instruments, Malvern, UK).

A suspension of small liposomes of ˜100 nm diameter was obtained (˜80-130 nm size distribution).

### Folate Receptor Targeted Liposomes:

Folate-derivatized ²⁰⁰⁰PEG-DSPE was synthesized as described by Gabizon et al. [Gabizon A, Horowitz A, Goren D, Tzemach D, Mandelbaum-Shavit F, Qazen M, and Zalipsky, S. Targeting folate receptor with folate linked to extremities of poly(ethylene glycol)-grafted liposomes: in vitro studies. Bioconjugate Chemistry 10 (2):289-98, 1999].

The folate-lipophilic conjugate (Folate-²⁰⁰⁰PEG-DSPE, MW=4051) was added to a fraction of the pre-formed liposomes at 0.5 molar ratio relative to the total phospholipid content. The Folate-²⁰⁰⁰PEG-DSPE was weighed in dry form, suspended in the liposome containing buffer and incubated at 45° C. for 2 hours with shaking for incorporation in the lipid bilayer. Then, the liposome suspension was cooled and centrifuged (10 min. 3000 rpm) to remove any precipitate of non-incorporated Folate-²⁰⁰⁰PEG-DSPE.

Folate-²⁰⁰⁰PEG-DSPE liposome content was determined spectrophotometrically at 284 nm after disruption of the liposomes by dilution 1:10 in 3% sodium dodecyl sulfate (SDS) as described previously [Gabizon et al., 1999, ibid.].

### LH-RH Targeted Liposomes:

LH-RH conjugation to carboxylated ^{2k}PEG-DSPE procedure is based on Dharap et al. methodology [Dharap S. S., Qiu B., Williams G. C., Sinko P., Stein S. Minko T. Molecular targeting of drug delivery systems to ovarian cancer by BH3 and LHRH peptides. Journal of Controlled Release 91: 61-73 (2003)].

Specifically, the sequence of the native LH-RH peptide is modified to provide a reactive amino group only on the side chain of a lysine residue, which replaced Gly at position 6 to yield the super-active, degradation-resistant Lys-6-des-Gly-10-Pro-9-ethylamide LH-RH analog (Gln-His-Trp-Ser-Tyr-Dlys-Leu-Arg-Pro-NHEt). The peptide is reacted with DSPE-PEG-NHS in DMF, purified by HPLC and characterized by mass spectrometry and ¹H-NMR.

### Cytotoxicity:

The cytotoxicity of free alendronate and of Folate targeted or non-targeted liposomal alendronate was determined in two folate receptor (FR)-upregulated human cell lines, KB and IGROV-1.

Cytotoxicity was assayed using varying concentrations of drug (0.1-200 μM) under standard 72 hr, continuous exposure, in 96-multiwell assays. Growth rate was assessed colorimetrically based on methylene blue staining or using the Promega MTS kit, and data was be obtained with an automatic plate reader and IC50 values were determined.

In addition, the cytotoxicity of free alendronate and of LH-RH-targeted and non-targeted liposomal alendronate is determined as described above for the Folate targeted and non-targeted liposomal alendronate.

### Toxicity:

Liposomal alendronate was injected i.v. to four Balb/C mice, at a starting dose of 20 μg/mice. The dose was escalated by doubling the dose every 14 days up to a dose of 320 μg/mouse. The toxicity of liposomal alendronate was determined from the daily weight measurements and observation of the mice throughout the experimental period.

### Results:

### Liposomal Formulation:

The content of a typical preparation of the targeted liposome was determined to be as follows:
Phospholipid (PL) concentration ˜30 μmol/ml,
Final bisphosphonate concentration of ˜1 mg/ml with a bisphosphonate/PL mole ratio of ˜0.10.
Average particle size of 80-130 nm (for extruded liposomes).

### Cytotoxicity:

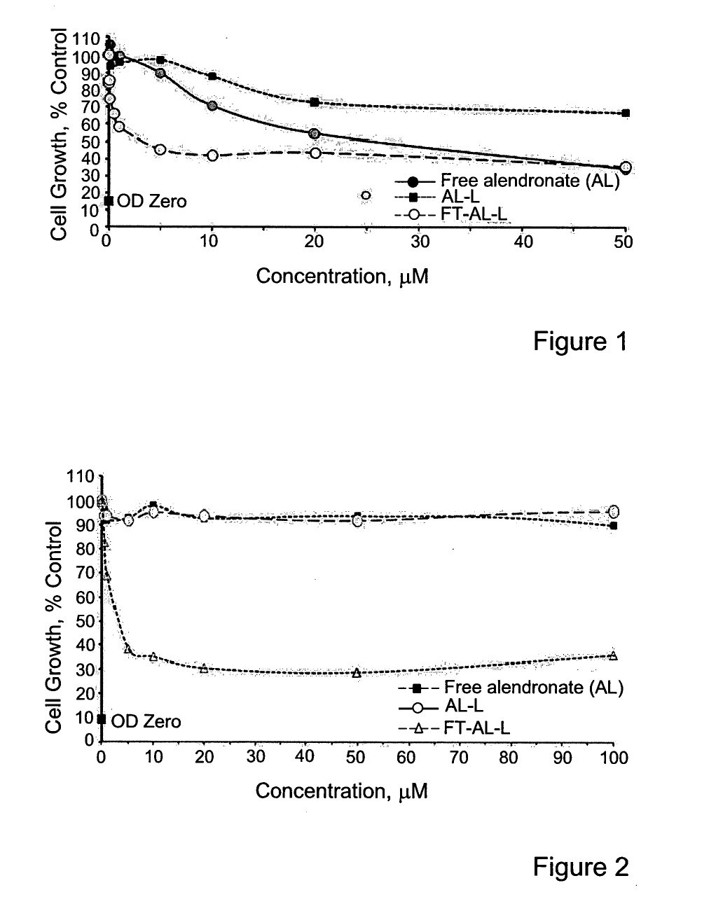

The cytotoxicity of free alendronate and of Folate targeted and non-targeted liposomal alendronate was evaluated in two FR-upregulated human cell lines IGROV-1 (FIG. 1) and KB (FIG. 2).

As shown in FIG. 1 and FIG. 2 and in the following Table 2, the folate targeted liposomal alendronate was significantly more potent than the free alendronate or the non-targeted liposomal alendronate, in both cell lines, as observed by the increased cytotoxicity of the folate targeted liposomal alendronate against the two tested cancer cell lines.

TABLE 2
IC₅₀ of free alendronate and of Folate targeted and non-targeted liposomal

| | | IC₅₀ μM | IC₅₀ μM |
|---|---|---|---|
| Cell type | Treatment | (−) folate | (+) folate |
| IGROV-1 | Free Alendroante | 26.5 | |
| | Liposomal Alendroante | >50 | 3.25 |
| KB | Free Alendroante | 157 | |
| | Liposomal Alendroante | >200 | 5.6 ± 1.2 |

### Toxicity:

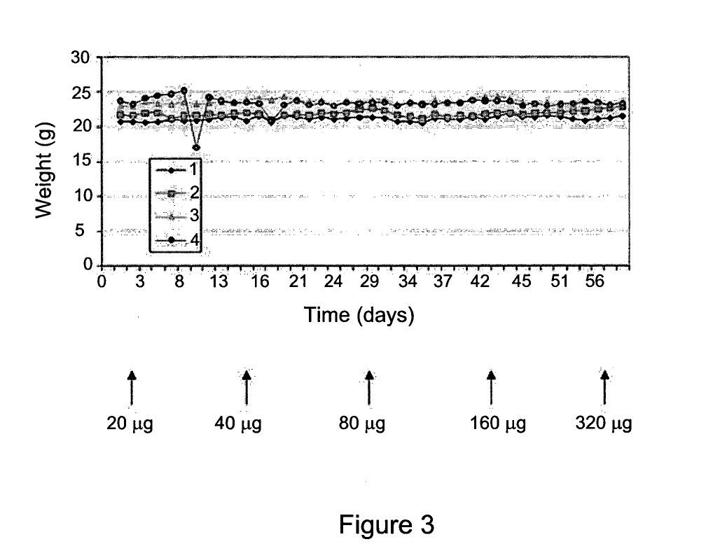

The maximal tolerated dose (MTD) of liposomal alendronate was evaluated after i.v. injection of escalating doses of the liposomal drug, staring at a dose of 20 μg/mouse (FIG. 3). The dose was doubled every 14 days up to a dose of 320 μg/mouse. The maximal cumulative dose of liposomal alendronate was determined at a total of 300 μg/mouse. From results with another liposomal bisphosphonate (unpublished data) it is expected that the toxicity of the folate-targeted liposomal alendronate to be the same as with the non-targeted liposomal formulation.

1-30. (canceled)

31. A targeted liposome comprising a membrane and an intraliposomal core, the membrane comprising at least one liposome forming lipid and a targeting moiety exposed at the membrane's outer surface; and the intraliposomal core comprising encapsulated therein least one N-containing bisphosphonate.

32. The targeted liposome of claim 31, wherein the mole:mole ratio between the N-containing bisphosphonate and the lipid is between 0.1 and 1.5.

34. The targeted liposome of claim 31, wherein the liposome comprises a single liposome forming lipid or a combination of liposome forming lipids, the single lipid or combination of lipids having a Tₘ equal or above 30° C.

35. The targeted liposome of claim 31, wherein the liposome's membrane comprises a cholesterol.

36. The targeted liposome of claim 35, wherein the amount of the cholesterol in the liposome's membrane is such that the lipid/cholesterol mole:mole ratio of the liposomes is in the range of between about 75:25 and about 50:50.

37. The targeted liposome of claim 31, wherein the liposome's membrane comprises a lipopolymer.

38. The targeted liposome of claim 31, wherein the liposome has a diameter in the range of 80-130 nm.

39. The targeted liposome of claim 31, wherein the liposome is a unilamellar vesicle.

40. The targeted liposome of claim 31, wherein the N-containing bisphosphonate is selected from the group consisting of alendronate, pamidronate, neridronate, olpadronate, ibandronate, risedronate and any physiologically acceptable salt thereof.

41. The targeted liposome of claim 37, wherein the targeting moiety is conjugated to the lipopolymer.

42. The targeted liposome of claim 41, wherein the N-containing bisphosphonate is alendronate and the targeting moiety is folate.

43. A method of treatment comprising administering to a subject in need of treatment an amount of targeted liposomes comprising a membrane and an intraliposomal core, the membrane comprising at least one liposome forming lipid and a targeting moiety exposed at the membrane's outer surface; and the intraliposomal core comprising encapsulated therein least one N-containing bisphosphonate.

44. The method of claim 43, wherein the mole:mole ratio between the N-containing bisphosphonate and the lipid is between 0.8 and 1.3.

45. The method of claim 43, wherein the liposome comprises a single liposome forming lipid or a combination of liposome forming lipids, the single lipid or combination of lipids having a Tₘ equal or above 30° C.

46. The method of claim 43, wherein the liposome's membrane comprises a cholesterol.

47. The method of claim 46, wherein the amount of the cholesterol in the liposome's membrane is such that the lipid/cholesterol mole:mole ratio of the liposomes is in the range of between about 75:25 and about 50:50.

48. The method of claim 43, wherein the liposome's membrane comprises a lipopolymer.

49. The method of claim 43, wherein the liposome has a diameter in the range of 80-130 nm.

50. The method of claim 43, wherein the liposome is a unilamellar vesicle.

51. The method of claim 43, wherein the N-containing bisphosphonate is alendronate and the targeting moiety is folate.

52. The method of claim 43, wherein the targeted liposomes are administered to the subject in need thereof by injection.

53. The method of claim 43, for treatment of a proliferative disease or disorder.

54. The method of claim 53, for the treatment of secondary bone cancer.

55. A pharmaceutical composition comprising as active ingredient targeted liposomes comprising a membrane and an intraliposomal core, the membrane comprising at least one liposome forming lipid and a targeting moiety exposed at the membrane's outer surface; and the intraliposomal core comprising encapsulated therein least one N-containing bisphosphonate.

56. The pharmaceutical composition of claim 55, wherein the N-containing bisphosphonate is alendronate and the targeting moiety is folate.
